(19) United States
(12) Patent Application Publication
(10) Pub. No.: US 2004/0092975 A1
(43) Pub. Date: May 13, 2004

(54) Anastomotic connection system

(76) Inventors: Amir Loshakove, Moshav-Bazra (IL); Ido Kilemnik, Herzelia (IL); Tanchum Feld, Moshav Merhavia (IL); Eitan Konstantino, Pleasanton, CA (US)

Correspondence Address: Reed Smith LLP, 29th Floor, 599 Lexington Avenue, New York, NY, 10022-7650, US

(21) Appl. No.: 10/402,375

(22) Filed: Mar. 27, 2003

Related U.S. Application Data
(63) Continuation of application No. PCT/IL01/00903, filed on Sep. 25, 2001.
(63) Continuation-in-part of application No. PCT/IL01/00600, filed on Jun. 28, 2001.

(30) Foreign Application Priority Data
Mar. 19, 1999 (IL) 129067

(51) Int. Cl. A61B 17/08

(52) U.S. Cl. 606/153

(57) ABSTRACT

A delivery system for an anastomosis connector, comprising: a body including a handle for applying force; a capsule adapted to interlock with said body and for carrying a connector, wherein said force is transferred by said interlocking to deploy said connector; and an extension, adapted to be connected between said body and said capsule, thereby extending a reach of said delivery system.

### RELATED APPLICATIONS

The present application is related to the following PCT publication and applications and is a continuation-in-part thereof: PCT/IL01/00600, PCT/IL01/00266, WO 01/41624, PCT/IL01/00074, WO 01/41623, WO 00/56226 and WO 00/56228 the disclosures of which are incorporated herein by reference.

### FIELD OF THE INVENTION

The present invention relates generally to the field of creating anastomotic connections, especially between blood vessels.

### BACKGROUND OF THE INVENTION

Several types of methods of connecting a graft to a blood vessel have been previously suggested, including, for example creating an anastomotic connection using a single connector and creating a connection using a plurality of surgical clips. In a side to end connection, an incision or a hole is usually formed in the side vessel and the end of the end vessel attached to the incision or hole.

After the anastomotic connection is completed, one or both of the blood vessels may change in diameter, for example, a saphaneous vein naturally increases in diameter, due to the increase pressure inside the vein. In addition, forces acting on the side blood vessel may extend any incision or hole formed in the vessel, potentially causing a leakage of blood.

### SUMMARY OF THE INVENTION

An aspect of some embodiments of the invention relates to an anastomotic connector, a set of clips for forming an anastomotic connection and/or an anastomosis delivery system, having a special design, for example a reinforcement and/or a motion limiter, at parts that correspond to high tension portions of the anastomotic connection, to prevent damage to a target blood vessel. Thus, in one embodiment of the invention, some clips are more loosely coupled (if at all coupled) than other clips.

In an exemplary embodiment of the invention, the anastomotic connection is characterized by including a plurality of tissue attachment points, for example spikes of a connector or individual clips, which attachment points can move relative to each other, for example, expand. In an exemplary embodiment of the invention, pairs of attachment points that are at ends of an incision are coupled together to reduce their relative motion. In the example of clips, a double, angled, clip is optionally provided for the incision ends.

In an alternative embodiment of the invention, each tissue attachment point is defined by an apertured segment and the apertured segments are not substantially distorted when the connector as a whole is distorted, for example to increase in radius.

An aspect of some embodiments of the invention relates to an anastomotic connection adapted to conform to blood vessel geometry and/or allow changes in blood vessel diameter. In an exemplary embodiment of the invention, the connector comprises a ring that interconnects a plurality of substantially independent clip elements. A virtual ring is defined by the clamp points of the clips (when closed). In an exemplary embodiment of the invention, the virtual ring and the ring are not congruent rings. Optionally, the rings are offset in the plane of the ring and/or along a direction perpendicular to the plane of the ring. Alternatively or additionally, the rings have different diameters. Alternatively or additionally, the ring has a different degree of obliqueness and/or orientation than the virtual ring.

In an exemplary embodiment of the invention, the differences in ring geometries cause the clips to be twisted relative to the ring. Alternatively or additionally, each clip can move separately, so that the clamp points define a non-planar ring that better conforms to the side blood vessel geometry. Alternatively or additionally, if the blood vessels change in geometry, the degree of twisting of each clip can change. In an exemplary embodiment of the invention the clips are designed to have a preferential twist direction, thus encouraging a desired blood vessel conformance. In some embodiments of the invention, the interconnection ring provided a minimum degree of flexibility while still imposing an flexible limitation on the relative positions and/or orientations of the clip elements. Optionally, the ring is thin or twist joints interconnect the clips and the ring, to allow the twisting.

In an exemplary embodiment of the invention, the clips are interconnected by straight segment. Alternatively, the clips are interconnected by bent segments, which allow radial expansion and/or support twisting.

In an exemplary embodiment of the invention, an anastomotic connector comprises a plurality of clips and a plurality of tissue pullers that pull vascular tissue into the clip.

In another exemplary embodiment of the invention, the connector is a base-plate type connector in which the base plate is flexible to allow conformance of base plate to the side blood vessel.

In an exemplary embodiment of the invention, the individual clips or portions of the base plate, even if apertured, do not substantially distort when the connector as a whole is distorted.

An aspect of some embodiments of the invention relates to a ring anastomotic connector in which a resilient ring interconnects a plurality of independently-patent tissue holders, for example clips or hook tips each of which can clamp together two blood vessel walls, against force. In an exemplary embodiment of the invention, the ring defines a desired anastomosis radius. The ring may be resiliently or plastically deformed to support a greater radius if the blood vessel expands. Alternatively or additionally, the ring may resiliently urge the radius towards a certain size. Different resiliency values may be used for different blood vessels, in which different post-bypass behaviors are expected. In some embodiments of the invention, a similar design is used for hole closure devices, for example to allow for distortion of the blood vessel.

An aspect of some embodiments of the invention relates to clips including an aperture for the passage of one or two tissue pullers. The clips may include one or more spikes to engage blood vessel tissue. Optionally, the aperture also accommodates a clip holder for holding the clip during tissue pulling.

Alternatively or additionally, the aperture is used to hold the clip in place during deployment or for guiding a clip deforming element.

An aspect of some embodiments of the invention relates to a puller-pair geometry, in which two pullers, each designated for a different blood vessel are arranged to be coaxial. In an exemplary embodiment of the invention, one puller fits along a slot defined in the other puller.

In an alternative embodiment of the invention, an anastomotic connector comprises an apertured base, through which a plurality of hooked pullers extend. Optionally, each aperture has associated therewith one or more tabs that cooperates with a geometry of the puller to prevent disengagement of the puller once the puller is retracted through the aperture.

An aspect of some embodiments of the invention relates to a base-plate type anastomosis connector that allows sutures to be added to correct defects in an anastomosis. In an exemplary embodiment of the invention, the base plate is apertured and/or is sparse (i.e., does not fill space), to allow room for insertion of a needle therethrough. Alternatively or additionally, the base plate is made of a piercable material. Optionally, the base plate is flexible to allow distortion of the base plate and/or anastomotic connection during stitching.

An aspect of some embodiments of the invention, relates to the design of a puller engagement element. In an exemplary embodiment of the invention, the element includes a tab that prevents disengagement of the puller once the puller is retracted. In an exemplary embodiment of the invention, the tab has curved tab geometry, to allow a greater length of a tab in a limited space. The length, coupled with a sufficient tab thickness and/or metallurgical treatment, optionally provide sufficient strength, resilience and/or range of motion to the tab. One, two or more tabs may be provided.

An aspect of some embodiments of the invention relates to a puller engagement element that includes a bar, which bar is engaged by a hook tip of the pullers. In an exemplary embodiment of the invention, the aperture is defined as a pair of openings separated by a short segment, such as a bar. The puller extends through one opening and, when retracted, the tip of the hook of the puller extends into the other opening.

An aspect of some embodiments of the invention relates to a method of holding a base part of an anastomotic connector during deployment. In an exemplary embodiment of the invention, at least two hinged tabs hold the base portion against a delivery system. When the base portion is to be released, the tabs are released and allowed to rotate, so they do not block the motion of the base plate. In an exemplary embodiment of the invention, the tabs are held at an outer section thereof and prevented from rotation by an element that hooks an aperture nearer an inner section thereof. The tabs are released by retracting the element so that it pulls through the aperture and releases it.

An aspect of some embodiments of the invention relates to an anastomotic connector having long pullers, which pullers are distorted to assist in mounting a graft on the connector. In an exemplary embodiment of the invention, the pullers include recurved tips, that curve back 180°. In an exemplary embodiment of the invention, the pullers are bent out and then back, so that the tips point radially out at about 90° to the axis of the connector. The graft is mounted, for example, by pulling its lip over the tips. In an exemplary embodiment of the invention, after the graft is mounted, the pullers are released to straighten. Alternatively or additionally, the extra length of the pullers is compensated for by an extra long retraction, during deployment.

An aspect of some embodiments of the invention relates to an extension mechanism for an anastomosis delivery and/or hole punching system. In an exemplary embodiment of the invention, a same system can be used for open chest, for keyhole and/or for endoscopic surgery. In an exemplary embodiment of the invention, the delivery system includes at least one axial rod (which may be hollow) that is advanced, retracted and/or rotated to effect the operation of the system. In an exemplary embodiment of the invention, the rod(s) is formed from two parts that interlock to provide the required coupling. To extend the delivery system, the two parts are disconnected and an extension piece having matching interlock mechanism inserted. Thus, for example, an anastomosis delivery system in which the pullers are retracted into a base part has the pullers coupled to an initial rod and an extension rod coupled to the initial rod. The pulling and sequencing mechanism is optionally located in the delivery system handle.

In an exemplary embodiment of the invention, an anastomosis delivery mechanism is provided as a modular capsule that can be attached to the delivery system and/or an extension. Optionally, a same drive mechanism is provided for punching and for anastomosis delivery, so that a same device can be used for both, except that a punching capsule is replaced by a delivery capsule. Both capsules, for example, may be activated by retraction and/or rotation of the rod.

Optionally, the capsule includes a lever, for example a rotating lever, for advancing and/or retracting the pullers, at least as part of an insertion process. In an exemplary embodiment of the invention, the lever locks in at least one and optionally two puller positions. Such a lever may also be supplied as a separate control in a non-capsule embodiment of the invention.

Optionally, the extension is flexible, rather than rigid as in other embodiments. Optionally, a channel for a camera and/or light source is provided in the extension, for assisting endoscopic surgery.

In an exemplary embodiment of the invention, the delivery system is a split system in which retraction of the rod causes the system to split and release a delivered blood vessel. In an exemplary embodiment of the invention, the end of the delivery system is coupled to the extension piece in a manner that keeps the two connected even when splitting is performed, for example, via the rod interlock or by providing an interlock between the bodies of the end and the extension.

An aspect of some embodiments of the invention relates to an anastomosis delivery system including at least one hinge and/or distortable portion, for use along non-straight path.

In an exemplary embodiment of the invention, the delivery system includes a flexible cable for transferring force (retracting force) from the delivery system handle to the connector. In one embodiment of the invention, the delivery system is hinged, for example in one, two, three or more dimensions. In another embodiments of the invention, the delivery system is flexible over its entire length, like a goose-neck.

An aspect of some embodiments of the invention relates to a side cutter for a blood vessel. In an exemplary embodiment of the invention, the side cutter includes an L shaped element having a sharpened tip. The tip is poked into a blood vessel and one arm of the L inserted into the blood vessel following the tip. The L element is optionally rotated so that its arm is parallel to the vessel axis. The L element is then retracted relative to a base, providing cutting action by an optional sharpened inner lip on the L and/or shearing action against the base. The base is optionally sharpened. The base may be provided on one sides of the L element or it may sandwich the L element. Optionally, the cutting arm of the L is parallel to the base, alternatively, the arm may be inclined towards the base or away from the base, relative to the axis of the cutter. In an exemplary embodiment of the invention, the cutting arm is flexible and twisted in towards the plane of the base.

An aspect of some embodiments of the invention, relates to a device for removing a connector from a blood vessel, for example, after mounting of a graft on the connector or after completing an anastomosis. In an exemplary embodiment of the invention, the connector comprises a ring around one blood vessel (or graft) and a plurality of spikes that extend from the ring (e.g. or through the ring) to penetrate a second blood vessel with sharp tips of the spikes. In an exemplary embodiment of the invention, the device comprises at least one pincer that is adapted to engage a single spike at the ring and then retract the spike relative to the ring. Optionally, the ring serves to straighten the spike as it is retracted. Optionally, the device does not block the motion of a tab part of the ring, which tab part is designed to prevent retraction of the spike under normal conditions. Alternatively, the device includes a protrusion that bends the tab out of the way. Alternatively, the pincer also engages and pulls back the tab.

There is thus provided in accordance with an exemplary embodiment of the invention, an anastomotic connector comprising:

a plurality of clip segments; and

a plurality of twistable resilient segments that interconnect the clip segments. Optionally, said segments are bendable out of a plane defined by said clip segments. Alternatively or additionally, a resilience of said attachment segments is defined to control a diameter changing behavior of said connector.

Optionally, said clip segments do not penetrate target tissue when the clip closes. Alternatively, said clip segments do penetrate target tissue when the clip closes, but do not transfix said tissue.

There is also provided in accordance with an exemplary embodiment of the invention, an anastomotic connector comprising:

a plurality of clip segments each defining a clip contact area at which opposite sides of the clip engage tissue; and

a plurality of attachment segments that interconnect the clip segments, wherein said attachment segments lie in a first circumference and said contact areas lie in a second circumference and wherein said two circumferences are not the same. Optionally, a resilience of said attachment segments is defined to control a diameter changing behavior of said connector. Alternatively or additionally, said circumferences are not on a same plane.

There is also provided in accordance with an exemplary embodiment of the invention, an anastomotic connector comprising:

a plurality of connection segments each defining a contact area between the segment and a target blood vessel; and

a plurality of attachment segments that interconnect said connection segments and limit relative motion of the connection segments, wherein some of said attachment segments limit relative motion of said connection segments more than the motion of other connection segments is limited. Optionally, said connection segments each comprises an apertured segments through which a puller that engages the target vessel can be advanced and retracted. Alternatively or additionally, the connector comprises a base plate, and said connection segments each comprises an apertured area the base plate. Alternatively or additionally, said connection segments each comprises a clip element. Alternatively or additionally, the connector is designed to limit relative motion to a greater degree for areas of the target vessel that are expected to be under a higher degree of strain. Alternatively or additionally, said connection segments are arranged in the form of an ellipse and relative motion between segments is reduced at narrow ends of the ellipse.

There is also provided in accordance with an exemplary embodiment of the invention, an anastomosis connector comprising:

a base plate having a plurality of apertures defined therein; and

a set of pullers adapted to pass through at least some of said apertures during deployment of said connector,

wherein said base palate is made spatially sparse enough to allow suturing of an anastomotic connection, using a needle, through the base plate.

There is also provided in accordance with an exemplary embodiment of the invention, an anastomosis connector comprising:

a plurality of clip segments, each clip defining an engagement volume in which the clip engages tissue;

a plurality of attachment segments interconnecting the clip segments; and

a set of tissue pullers each including at least one tissue engager,

wherein, said clips define a plurality of apertures adapted for extending said tissue pullers through the apertures such that retracting the pullers carries engaged tissue into said engagement volume of said clips. Optionally, said apertures are adapted for holding the connector using a connector holder. Alternatively or additionally, said tissue pullers comprises pairs of axially elongate pullers, each pair comprising two pullers co-axially disposed with respect to each other. Optionally, one of said pair of connectors is adapted to engage tissue of a target blood vessel and wherein another of said pair of pullers is adapted to engage tissue of a vessel on which said connector is mounted prior to completing said anastomosis. Alternatively or additionally, at least one of said pair of pullers has a tip that is wider than said aperture, such that retracting the puller closes the clip when said clip has a resting point on either side of said aperture.

There is also provided in accordance with an exemplary embodiment of the invention, an anastomosis connector comprising:

a base plate defining a plurality of apertures therein; and

a set of pullers adapted to pass through at least some of said apertures during deployment of said connector, each of said pullers including a tissue engaging tip,

wherein said apertures are arranged in pairs, one aperture for receiving a puller and one aperture for receiving the tip of the puller when the puller is retracted. Optionally, the connector comprises a bar around which said puller hooks, which bar defines a separation between said pair of apertures.

There is also provided in accordance with an exemplary embodiment of the invention, an anastomosis connector comprising:

a base plate defining a plurality of apertures therein; and

a set of pullers adapted to pass through at least some of said apertures during deployment of said connector,

wherein said each of said pullers comprises:

a tip; and

a tab section adapted to distort out of a plane of said puller when said puller is disconnected adjacent said tab.

There is also provided in accordance with an exemplary embodiment of the invention, a delivery system for an anastomosis connector, comprising:

a body; and

at least one slotted forward plate attached to said body, wherein said plate and said body define therebetween a receptacle for a base plate of an anastomosis connector and wherein said slots match apertures formed in said base plate for extension of tissue engagement spikes therethrough,

wherein said forward plate is adapted to bend out of the way of axial motion of said base plate, when said connector is deployed. Optionally, said forward plate is mounted on at least one hinge. Alternatively or additionally, said forward plate is divided into at least two coplanar plates. Alternatively or additionally, said forward plate is attached to said body via a distortable attachment.

There is also provided in accordance with an exemplary embodiment of the invention, a delivery system for an anastomosis connector, comprising:

a body including a handle for applying force;

a capsule adapted to interlock with said body and for carrying a connector, wherein said force is transferred by said interlocking to deploy said connector; and

an extension, adapted to be selectively connected between said body and said capsule, thereby extending a reach of said delivery system. Optionally, said extension is bendable.

There is also provided in accordance with an exemplary embodiment of the invention, a delivery system for an anastomosis connector, comprising:

a body including a handle for applying force;

a connector holder area defined at a distal end of said body and adapted for holding an anastomosis connector, wherein said force is transferred to said area for deploying said connector; and

a non-limp geometry changing elongate section bridging between said body and said area. Optionally, said elongate section is hinged. Alternatively or additionally, said elongate section is distortable. Alternatively or additionally, the system comprises a flexible cable for transferring said force between said handle and said area.

In an exemplary embodiment of the invention, said system is adapted for holding and deploying a two part connector comprising a plurality of tissue engaging elements that are retracted by said force during deployment, such that a base ring portion of the connector is engaged by the holder area and a tissue puller portion of the connector is retracted by said force.

There is also provided in accordance with an exemplary embodiment of the invention, a delivery system for an anastomosis connector, comprising:

a body including a handle for applying force;

a connector holder area defined at a distal end of said body and adapted for holding an anastomosis connector, wherein said force is transferred to said area for deploying said connector; and

a control for selectively advancing a plurality of tissue engaging elements from said connector holder area, said control being separate from said handle for applying force. Optionally, said control comprises a rotating knob. Alternatively or additionally, said control is mounted on a separate capsule element that includes said connector holder area. Alternatively or additionally, said tissue engaging elements form part of a connector. Alternatively or additionally, said tissue engaging elements form part of said delivery system.

There is also provided in accordance with an exemplary embodiment of the invention, a connector removal device for removing a spike that completes an anastomotic connection in a spike and base type connector, comprising:

at least one spike engager adapted to engage at least one spike of a deployed connector;

an axially elongate contra element axially movable relative to said spike engager and adapted to rest against a base portion of said connector while said spike engager retracts said spike away from said base portion by said axial motion. Optionally, said spike engager engages said spike by friction. Optionally, said spike engager comprises a split tube. Alternatively or additionally, said contra element comprises an overtube that fits over said spike engager and wherein said contra element has an inner diameter that clamps said spike engager shut when said engager is within said contra element. Alternatively or additionally, said spike engager engages a plurality of spikes at a time.

There is also provided in accordance with an exemplary embodiment of the invention, a method of mounting a graft on a generally cylindrical connector having a plurality of axially extending spikes, each with a curved tip at a distal end of the connector, comprising:

bending each spike such that the spike points inwards towards the axis and the tip points outwards away from the axis;

bringing a graft to said axis between the inward pointing spikes; and

mounting the graft on the spike points. Optionally, bringing comprises conveying the graft between the spikes from a proximal end of the connector to the spike tips. Alternatively or additionally, mounting comprises mounting the graft sequentially over the tips. Alternatively or additionally, bending comprises mounting the connector in a jig. Alternatively or additionally, bending comprises bending the spikes outwards and then bending inwards so that the bent part of the spikes define a diameter greater than that of unbent spikes.

There is also provided in accordance with an exemplary embodiment of the invention, a jig for holding a spiked connector in a configuration in which the spikes bend inwards, comprising;

a body defining an axial channel for receiving a connector; and

a plate attached to an end of said body and defining a plurality of trans-axial channels for receiving spikes of said connector, said plate defining a hole in its center for receiving a graft. Optionally, said plate comprises a slotted disk. Alternatively or additionally, the jig comprises at least one rotatable disk underlying said plate for selectively defining slots along which said spikes can be conveyed when twisted. Alternatively or additionally, the jig comprises at least one rotatable slotted disk overlying said plate for selectively aligning slots of said disk with slots of said plate. Optionally, said slots in said disk are arranged so that said selective alignment can be sequential for different ones of said slots of said plate.

There is also provided in accordance with an exemplary embodiment of the invention, a blood vessel cutter for forming an aperture in a blood vessel, comprising:

a first section having a cutting face; and

a second section having a cutting face matching said cutting face of said first section and axially movable relative thereto to provide shearing cutting action,

wherein, said second section comprises a pointed tip adapted for piercing a blood vessel; and

wherein said second section is flexible and said pointed tip is twisted towards a plane of said first section, such that when said first section is moved towards said second section, said first section pushes said second section out of its way. Optionally, said cutting face of said second section is sharp. Alternatively or additionally, said cutting faces are not parallel to each other in a plane generally common to the two cutting faces. Alternatively or additionally, said second cutting face is not perpendicular to said axial motion. Alternatively or additionally, said pointed tip points away from said first plane.

### BRIEF DESCRIPTION OF THE FIGURES

Non-limiting embodiments of the invention will be described with reference to the following description of exemplary embodiments, in conjunction with the figures. The figures are generally not shown to scale and any measurements are only meant to be exemplary and not necessarily limiting. In the figures, identical structures, elements or parts which appear in more than one figure are preferably labeled with a same or similar number in all the figures in which they appear, in which:

FIG. 1 illustrate an anastomosis clip set mounted on a clip deployment system that includes a plurality of pullers, in accordance with an exemplary embodiment of the invention;

FIG. 2 illustrates a delivery system for elastic clips, in accordance with an exemplary embodiment of the invention;

FIG. 3 illustrates a portion of a delivery system for plastic deforming of clips, in accordance with an exemplary embodiment of the invention;

FIG. 4 illustrates an alternative clip system in which clips face inwards rather than axially, in accordance with an exemplary embodiment of the invention;

FIG. 5 illustrates an alternative clip design, in accordance with an exemplary embodiment of the invention;

FIGS. 6A and 6B illustrate a clip-ring connector, in accordance with an exemplary embodiment of the invention;

FIGS. 7A-7E illustrate apertured-base anastomotic connectors, in accordance with an exemplary embodiment of the invention;

FIGS. 8A-8D illustrate an extendible delivery system in accordance with an exemplary embodiment of the invention;

FIGS. 8E-8H illustrate distortable and bendable delivery systems, in accordance with exemplary embodiments of the invention;

FIGS. 9A-9E illustrate an alternative embodiment of a capsule based delivery system, in accordance with an exemplary embodiment of the invention;

FIG. 10 illustrates a cutter for forming an opening in a target vessel, in accordance with an exemplary embodiment of the invention;

FIGS. 11A and 11B illustrate a connector and an associated graft mounting system, in which the connector is distorted to assist in mounting a graft thereon, in accordance with an exemplary embodiment of the invention; and

FIGS. 12A-12D illustrate a connector removing device, in accordance with an exemplary embodiment of the invention.

### DETAILED DESCRIPTION OF EXEMPLARY EMBODIMENTS

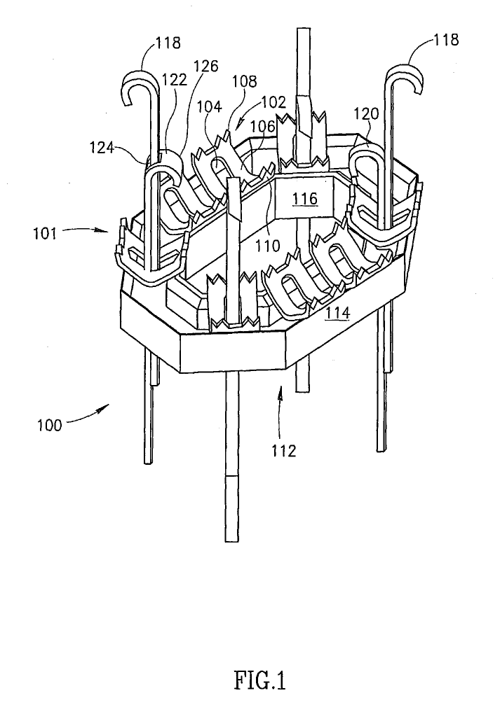

FIG. 1 illustrate an anastomosis clip set 101 mounted on a clip deployment system 100 that includes a plurality of pullers 118 and 120, in accordance with an exemplary embodiment of the invention.

An anastomotic connection is performed by inserting pullers 118 into an opening in a target side vessel and pulling the vessel wall into a plurality of clips 102 using the pullers. The tissue of an end vessel is also pulled into the clip using the same or other pullers. The clips are then closed, locking together the two blood vessels. Various configurations of pullers and blood vessels have been suggested in pervious applications of the present assignee. In the particular example shown, a pair of pullers may be provided for each clip, although not shown for clarity.

In an exemplary embodiment of the invention, one or both of the pullers are provided through an aperture 104 formed in a body 106 of each clip. In FIG. 1, not all clips 102 have pullers, while this is done for clarity of presentation, in some embodiments of the invention, not all clips are actually provided with one or both pullers.

In the particular embodiment shown, the clips rest on a holder 112 comprising an inner ring 116 and an outer ring 114. Closing the clips can be achieved, for example, by retracting the clips, for example using additional pullers (not shown) on either side of the clip. Alternatively, the pullers are used to close the clips. In one embodiment of the invention, the hooked tips of the pullers urge the tissue forcefully against body 106 of clip 102, so as to cause it to close. Alternatively, the puller tip may be wide enough to directly apply force to clip body 104. The puller may then be distorted (open) as it is further pulled out or it may be cut. One or both of the puller tips is optionally designed to penetrate the vessel tissue. Alternatively, they may be designed to not penetrate the tissue.

In an exemplary embodiment of the invention, the pullers are side by side, for example pullers 118 and 120. Alternatively, the pullers are coaxial, for example, a puller 122 has a slotted shaft 124 through which a puller 118 can fit. A tip 126 as shown is wide enough to distort clip 102.

The ends of the clips are optionally serrated, for example having four teeth 108 and 110 on either side of the clip. Fewer teeth (e.g., two) may be provided. Optionally, the teeth interlock (not shown). Alternatively, spikes that penetrate the vessel tissue to a significant depth and/or transfix one or more layers of tissue, are provided at the ends of the clips. Alternatively, the clips do not penetrate or do not transfix the blood vessels.

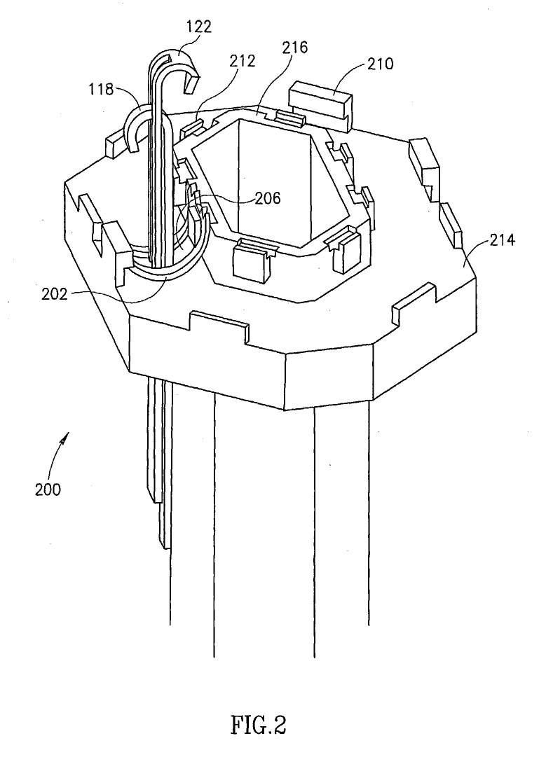

Alternatively to plastically deforming the clips, in an exemplary embodiment of the invention, the clips are self-deforming, for example, being elastic, super elastic of shape-memory. FIG. 2 illustrates a delivery system 200 for elastic clips, in accordance with an exemplary embodiment of the invention. Only a single clip and pullers is shown, for clarity, however, typically more than one clip is deployed at a time. An exemplary elastic clip 202, that can have the same geometry as clip 102 (e.g., with an aperture 206), except that it is self-deforming, is restrained from closing by a tab 212 and a tab 210. The tabs may hold the clip from its ends, for example from inside its aperture 206 as shown for tab 212 and/or from outside, for example as shown for tab 210. Alternatively the clip may be held from the side (and optionally released by rotation of the holder).

In an exemplary embodiment of the invention, a plurality of tabs 210 is mounted on an outer ring 214 and a plurality of tabs 212 is mounted on an inner ring 216. In use, pullers 122 and 118 (or only one puller) are used to retract tissue into clip 202 and then inner ring 216 is retracted and/or outer ring 214 advanced, to release the clip to self-deform to a closed configuration.

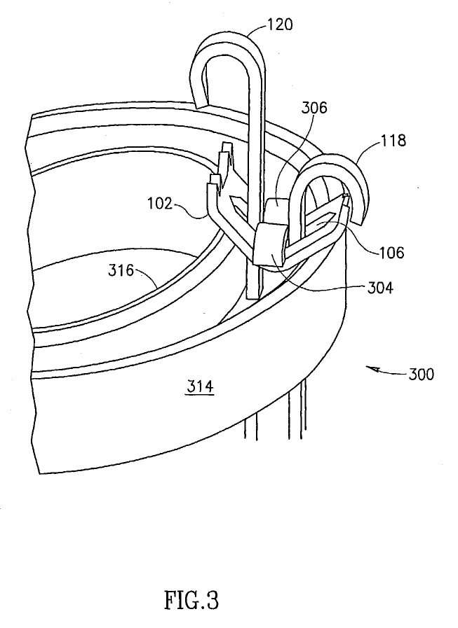

FIG. 3 illustrates a delivery system 300 for plastic deforming of clips 102, in accordance with an exemplary embodiment of the invention. Two clip holders 304 and 306 are shown for engaging the sides of clip 102. As shown, the holders are shaped like pullers and also pass through aperture 105. Optionally, the holders separate the two pullers. Alternatively or additionally, the holders are provided from outside of clip 102. In use, after pullers 118 and 120 pull tissue into clip 102, holders 304 and 306 retract the clip, which is bent by as the clip is longer (in its open configuration) than a space between a pair of rings 314 and 316 that hold it. Alternatively or additionally, the two rings advance towards each other to squeeze the clip shut. Optionally, the rings do not change in diameter, and supported on shafts that are not coaxial, so that when one ring is rotated the spacing between the rings changes.

Holders 304 and 306 are optionally removed by further retraction that deforms the shape of their tips.

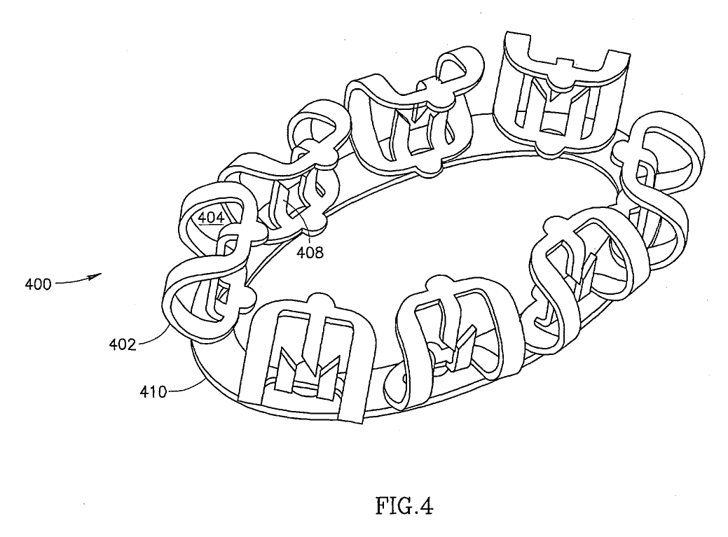

FIG. 4 illustrates an alternative clip system 400 in which a plurality of clips 402 face inwards rather than axially. In one example, clips 402 are always in a closed configuration as shown and the pullers (not shown), which are optionally provided through apertures 404 of clips 402, pull the tissue past one or more spikes 408 of clips 402. Alternatively, clips 402 are self-deforming and the pullers retract the tissue while the clips (but optionally not the spikes) are flat. The clips are then released to self deform to the configuration shown in FIG. 4. Optionally, a base ring 410 is attached to the clips and controls their relative positions and/or holds down one side of the clips. Optionally the ring is detached after the anastomosis is completed. Alternatively ring 410 remains in the body, the ring can be deformable, for example, as described below. In one example, a plurality of clip holders (not shown) urge clips 402 against ring 410 during deployment. Other clip orientations can also be provided.

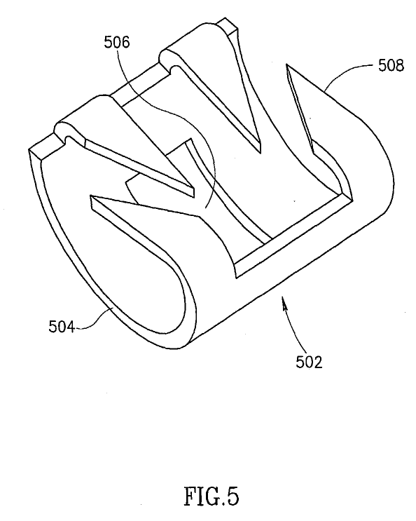

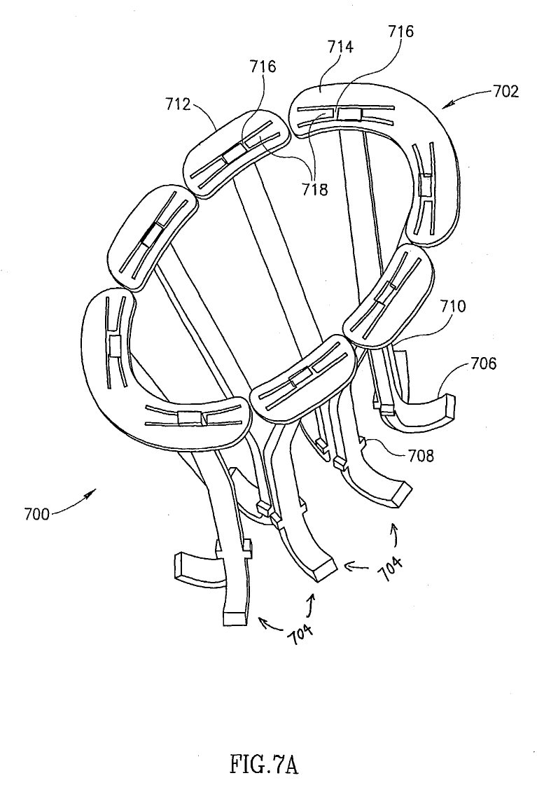

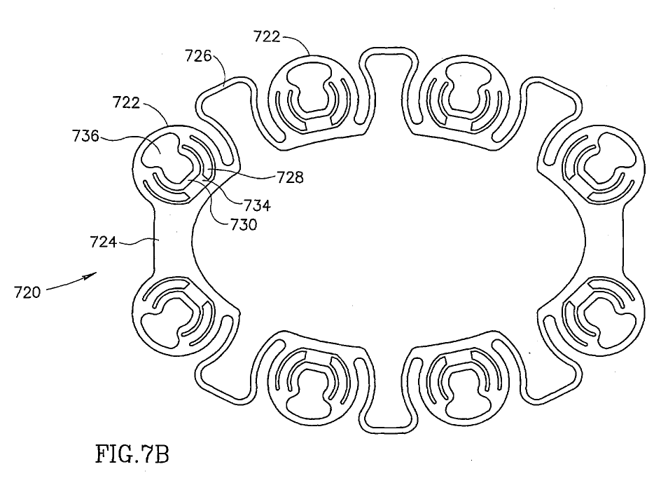

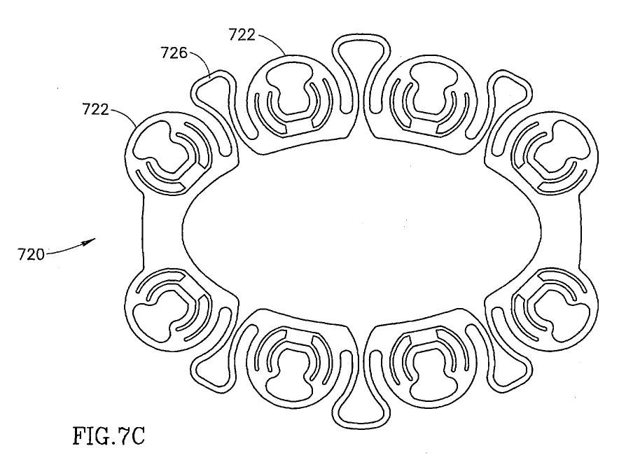

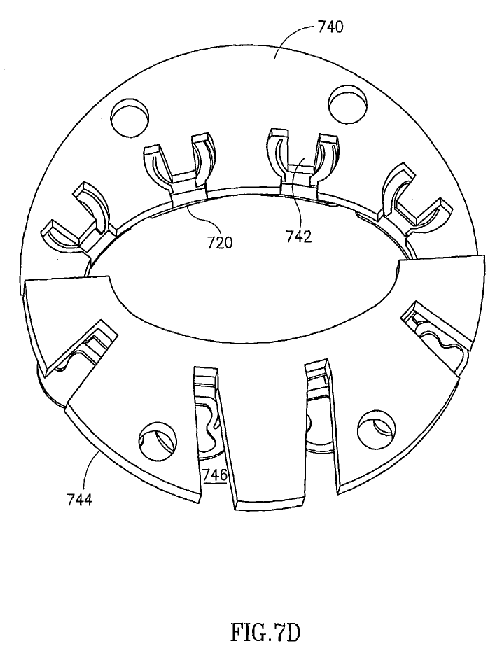

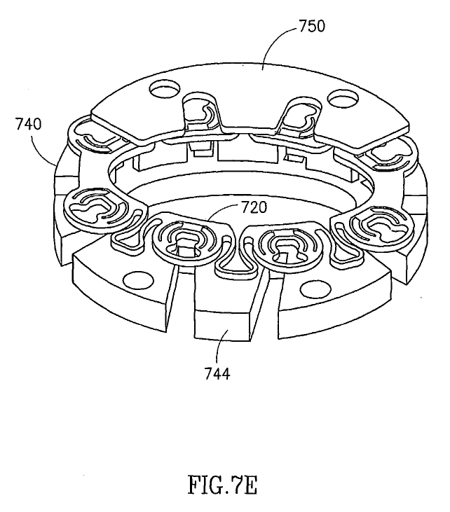

FIG. 5 illustrates an alternative clip design 502, in accordance with an exemplary embodiment of the invention. One or more spikes 508 of clip 502 can be, for example curved (as shown) or re-curved (as shown in FIG. 4). The size and shape of an aperture 506 in a body 504 of the clip can also vary. As shown below in FIG. 7, in some cases, the aperture is split into two parts.

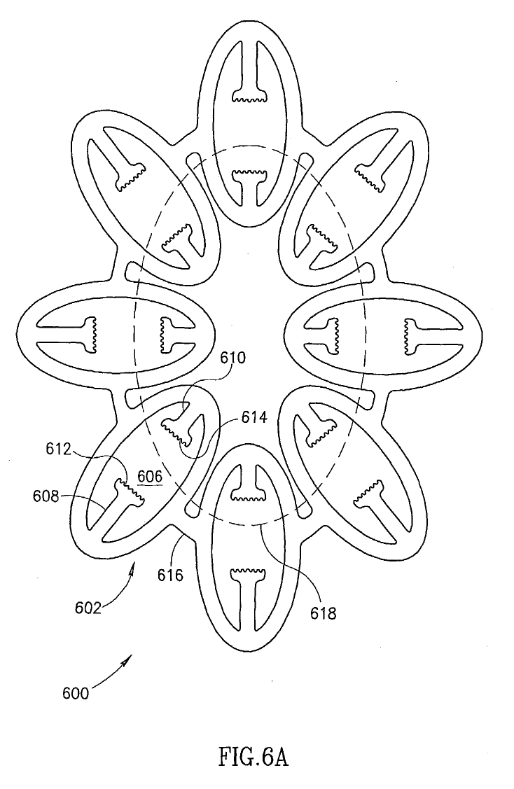

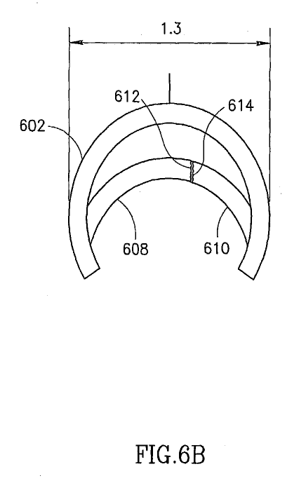

FIGS. 6A and 6B illustrate a clip-ring connector 600, in accordance with an exemplary embodiment of the invention.

Connector 600 comprises a plurality of individually patent clip elements 602 that are interconnected by a ring of short segments 616. In the particular embodiment shown, the clips are all of one type, but this is not essential. As shown the clips comprise an elliptical body 604 having two inward pointing extensions 608 and 610 that terminate in contact surfaces 612 and 614. Alternative clip designs, such as using spikes, using a unenclosed body or a non-elliptical body, may also be provided. Optionally, an aperture 606 defined by enclosing body 604 serves as a channel for one or more tissue pullers.

In an exemplary embodiment of the invention, a clip 602 is closed by bending, while the extensions which optionally also bend, meet at their tips, at surfaces 612 and 614. FIG. 6B shows a side view of such a closed clip. The curved shape can be achieved, for example, if the clip is self-deforming. A device similar to that of FIG. 2 is optionally used for deployment

In an exemplary embodiment of the invention, extensions 608 and 610 are designed to meet off-center from ring 610, along a virtual ring 618, which can have a varying diameter. If spikes are used instead of extensions, the spikes optionally include tissue stops to ensure that the clamped tissue meets at virtual ring 618. FIG. 6B shows the contact between surfaces 612 and 614.

In an exemplary embodiment of the invention, the closed clips twist and/or bend around ring 616, allowing the connector as a whole to better adapt to a blood vessel diameter change and/or to better conform to the surface of the side vessel and prevent over-straining of the side vessel.

Optionally, ring 616 is extendible, for example as described below if the segments are curved rather than straight.

FIGS. 7A-7E illustrate apertured-base anastomotic connectors, in accordance with an exemplary embodiment of the invention. In such a connector, a plurality of pullers 704 pull tissue towards a base plate 702 and then the pullers are sheared leaving only their tips 706 urging the tissue against the base plate. Both tips and base plate remain in the body. It should be noted, however, that the various features described below (e.g., flexible base plate) may also be applied towards clip-based devices (e.g., as a flexible clip ring), in which the tips do not remain in the body.

FIG. 7A illustrates a hybrid base-plate connector 700 in which some of the apertures of the base plate are provided as individual elements and some as mini-base plates including two or more apertures. The elements are optionally held together by the delivery system (e.g., the continuation of the pullers may meet) and/or by the blood vessel, after deployment. In the embodiment shown, a plurality of individual-aperture plates 712 each include an aperture 716 and a pair of tabs 718 to control the passage of a puller 704 therethrough. At the ends of the connector, a larger base-plate 714 having two apertures is provided. In an exemplary embodiment of the invention, the connector is inserted through an incision in a blood vessel. The larger base plate is provided to prevent or reduce relative motion of two tissue attachment points at the ends of the incision, which might extend the incision and cause a blood leak. As the connection process causes stress in the vessels, in some embodiments, the aperture plates are not interconnected, but the guides for retracting the pullers are interconnected to prevent motion at the ends of the incision.

In the exemplary embodiment shown, each puller 704 comprises a shaft 710 having protrusions 708 that match tabs 718. An optional puller shearing point (e.g., a thinning) is not shown. As described in other applications of the present assignee, the puller includes a tab that prevents the puller tip from falling out of the aperture. The tab cannot pass by the cutting plate (744, FIG. 7C) so further attempted retraction of the puller tears the puller, at the shearing point, which is, for example, a thinner portion of the puller shaft.

Optionally, the other aperture-plates are also interconnected, for example using a resilient spring which will allow relative radial and/or twisting motion of the aperture plates. An exemplary such base plate 720 is shown in FIG. 7B.

Base plate 720 comprise a plurality of apertures element 722 interconnected by resilient spring segments 726. Optionally, at the ends of the ring, relatively rigid connection segments 724 are provided.

Each aperture element 722 optionally comprises a bar 730 that separates the aperture into two parts, a passage aperture 734 for passage of the puller and a hook aperture 736 for receiving the tip of the puller. In an exemplary embodiment of the invention, the tip of the puller passes past the plane of bar 730. Optionally, the hook aperture is larger than the passage aperture, to compensate for freedom of movement of the tip. In other embodiments, the tip of the puller does not enter an aperture. The tip may penetrate the target tissue or not.

In an exemplary embodiment of the invention, one or more tabs 728 is provided to engage protrusions 708 and prevent the puller from falling out once it is retracted past the tabs. After deployment, the tabs themselves, or the tabs backed by the vessel wall, possibly prevent reverse motion of the tabs. Optionally, the tabs are curved, to allow a longer length in the limited space of aperture element 722.

In an exemplary embodiment of the invention, base plate 720 is made sparse to allow an anastomosis connection to be enhanced or corrected by passing a manual suture.

FIG. 7C shows base plate 720 in a radially contracted configuration, with spring segment 726 bend to allow aperture elements 722, which are generally not affected by the radial compression, to be closer together. Base plate 720 may be elastically or plastically deformed. In an exemplary embodiment of the invention, base plate 720 is used to control the deformation of the blood vessels of the anastomosis. In one example, base plate 720 is pre-stressed to a desired diameter which is smaller or lager than the current diameter. In another example, base plate 720 acts against undue expansion of the blood vessel. In an exemplary embodiment of the invention, the diameter of the anastomosis is selected to allow enough blood to flow both downstream (e.g., in a coronary vessel tree) and upstream (to possible collateral vessels).

In an exemplary embodiment of the invention, the circumference of base plate 720 increases or decreases by 5%, 10%, 20% or any smaller larger or intermediate percentage. Optionally, aperture elements 722 also provide a clip function, for example, as described above.

FIG. 7D is a partial cut-away view showing base plate 720 during exemplary deployment (some parts removed for clarity and some parts shown covering only part of base plate 720, instead of all the base plate). An optional spacer 740 is provided, that includes tabs 742 for preventing motion of bar 730 (not shown), while allowing motion of tabs 728 and passage of the puller. A cutting plate 744 having a plurality of apertures 746 provides a strong base against which the pullers can be sheared by retraction. FIG. 7E, also a partial cut-away view, shows the addition of an optional holder 750 which prevents base plate 720 falling into the body. After the anastomosis is started or completed, holder 750 is pulled back. In one example, holder 750 is hinged and it unfolds. In another embodiment, holder 750 is distorted by the retraction. In another embodiment, holder 750 is pulled out radially.

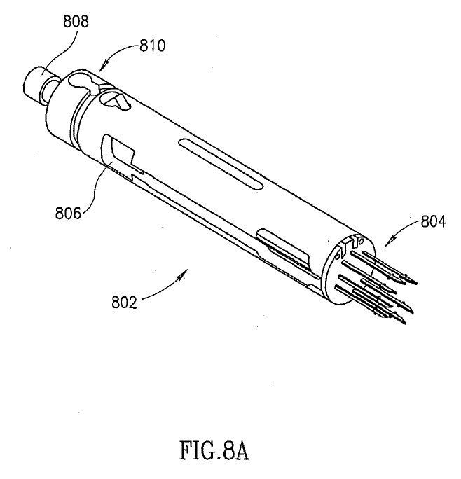

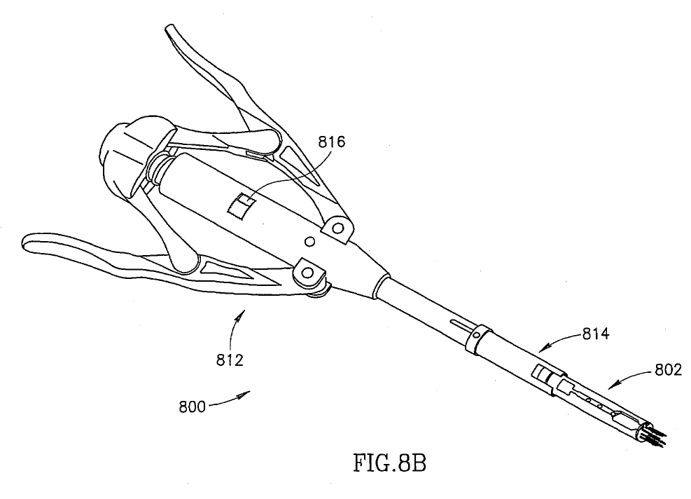

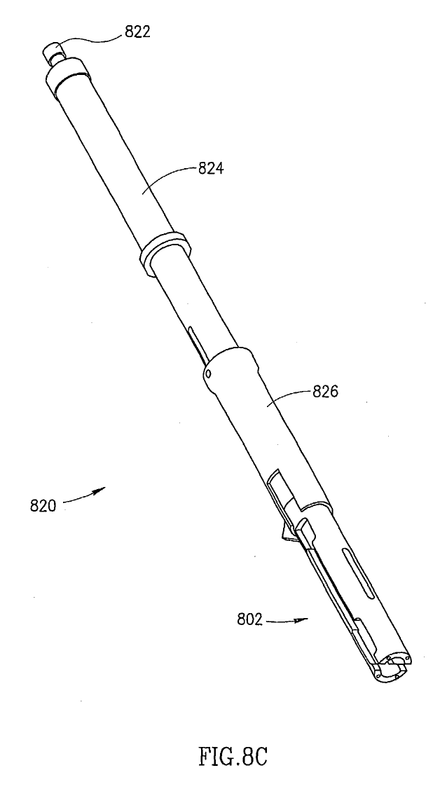

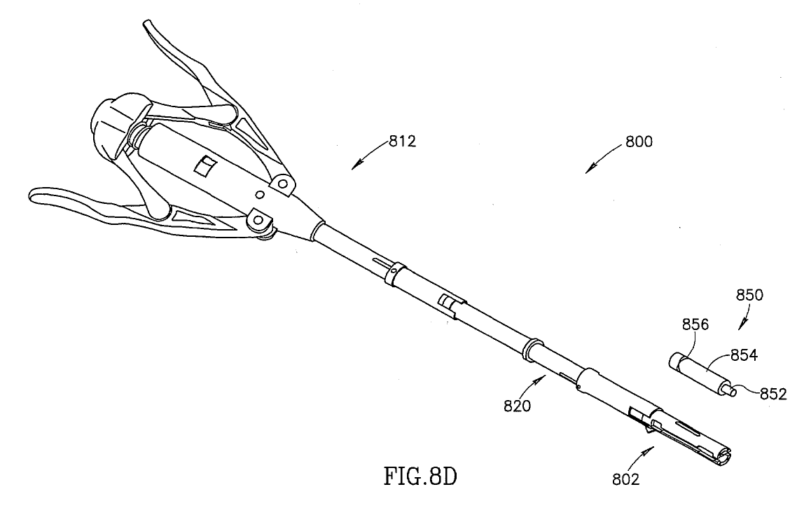

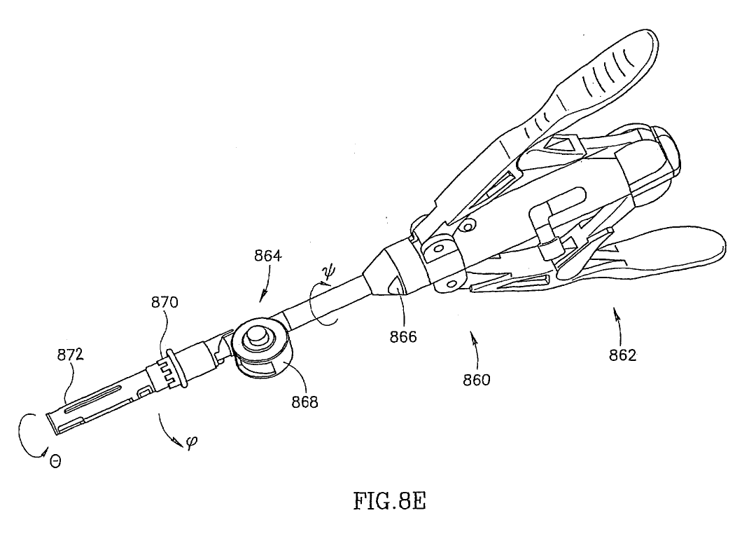

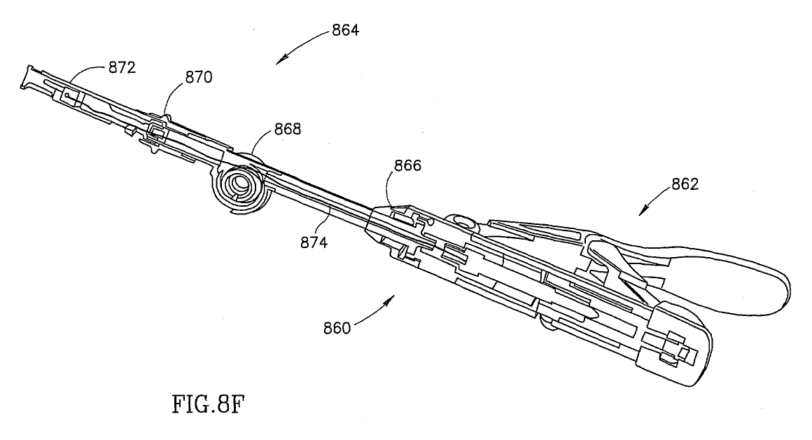

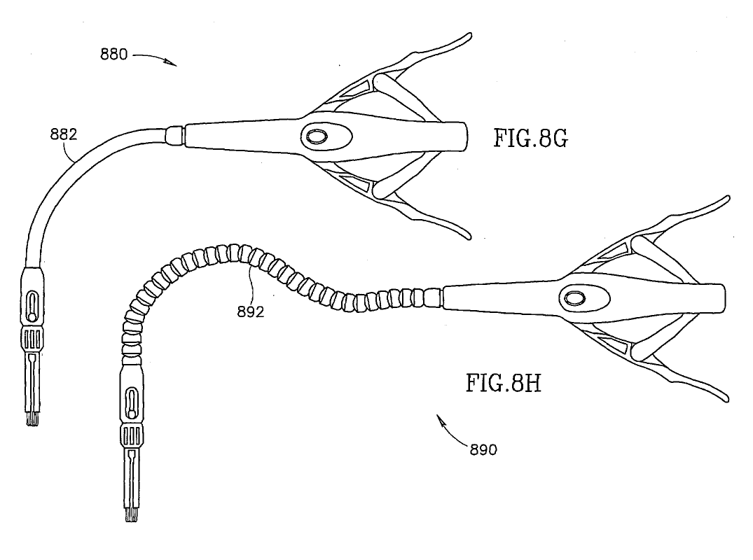

FIGS. 8A-8D illustrate an extendible delivery system 800 in accordance with an exemplary embodiment of the invention, in which the actual delivery of a connector is performed by a modular capsule 802. A similar design may be used for other types of anastomosis delivery systems and/or for other endoscopic activities, such as punching holes and suturing. A potential advantage of such a modular design is that a same system can be used for different surgical approaches and methods, for example, endoscopic, key-hole and open chest. Another potential advantage is that a single handle can be used for multiple parts of a procedure, reducing the size and/or number of components in a kit.

In an exemplary embodiment of the invention, any endoscopic tool that is activated using rotation, retraction or advancing of a rod can be made modular by providing a suitable coupling for the power and control mechanism. Typically, both power and control are provided by a same element.

FIG. 8A shows a connector capsule 802 including a connector 804 as described in FIGS. 7B-7E, an aperture 806 for insertion of a blood vessel and including an optional spilt for splitting the capsule for removal after the anastomosis is performed. A locking mechanism 810 is provided for locking to the rest of the delivery system and a rod extension 808 is provided to transfer retraction from the handle (FIG. 8B) to retract the pullers. Extension 808 may be polygonal or slotted, to support rotation. In other connector types, advancing of the connector or retraction of an over tube may be practiced instead, for example. Optionally, capsule 802 includes a power train for changing displacement amounts to allow a same handle to be used with different capsules that require different amounts and/or directions of motion. Alternatively or additionally, the handle includes such a power train and/or includes means for allowing both retraction and advancing.

FIG. 8B shows a complete system 800 including capsule 802 coupled via a coupler 814 to a handle segment 812. Optionally, a retraction indicator 816 is provided on the handle to indicate a degree of retraction. In an alternative embodiment, reference 816 indicates a locking switch.

FIG. 8C illustrates an extender 820 that fits between coupler 814 and capsule 802. A part of capsule 802 is also shown coupled to a coupler 826 that corresponds to coupler 814 in FIG. 8B. A shaft 824 serves to extend the reach of the device and terminates in a rod extension 822, corresponding to extension 808 of FIG. 8A. Shaft 824 may be rigid, or it may be partly or completely flexible or hinged, at one or more points along its length, for example as shown below in FIGS. 8E-8H. Optionally, shaft 824 is deformable, for example like a goose-neck. Optionally, shaft 824 includes a channel or slot (not shown) to act as a working channel, for example, to provide a camera, light source, material or tool to the tip of system 800. Such a channel is optionally provided also in handle 812 and/or in the capsule.

FIG. 8D shows a complete extended system.

Optionally, system 800 is used for an anastomotic connection in which leakage of blood is to be prevented after hole punching. In an exemplary embodiment of the invention, a shaft 850 including a homeostatic valve 856 (e.g., a leaflet valve) is used as a guide for punching and anastomosis delivery. In one exemplary implementation, a punch capsule is provided through a bore 854 of shaft 850. After the hole is punched, narrowing 852 is advanced over the punch into the holed blood vessel. The punch is then retracted and blood leakage is prevented or reduced by valve 856. A device delivery capsule (e.g., like capsule 802) is then provided through bore 854 and used to perform the anastomosis. Optionally, shaft 850 includes an axial tear line (not shown, for clarity) so that it can be torn off of the delivery system once capsule 802 is in place. Shaft 854 may be, for example, short, like a capsule or long enough to reach outside the body.

FIGS. 8E-8H illustrate distortable and bendable delivery systems, in accordance with exemplary embodiments of the invention.

FIG. 8E shows a delivery system 860 including a hinged extension 864. In the example shown, a separate capsule 872 is provided, for example the same capsule as capsule 802, describe above. Alternatively, the extension is integral with the capsule. In an exemplary embodiment of the invention, extension 864 is connected to a handle portion 862 of system 860 at a rotational hinge 866. A second rotational hinge 870, for example, integral with the capsule connection mechanism provides a second degree of rotation.

Optionally, a bending hinge 868 is provided between the two rotational hinges. In an exemplary embodiment of the invention, the total effect of the three hinges is to allow three angular degrees of freedom in positioning the tip of capsule 872.

FIG. 8F is a cut-through view of system 860 including a showing of a possible mechanism for transferring power from handle 862 to retract a connector device held in capsule 872. A cable 874 is substantially unaffected by the changes in geometry of extension 864, while still being able to retract a connector when force is applied to handle 862. In some embodiments, cable 874 is not taunt, until handle 862 is activated. Non-cable means, such as a chain or interlocking rails or rods may be used for transferring the force.

It should be appreciated that other numbers and/or types of hinges may be provided instead of as shown, possibly yielding fewer or greater degrees of freedom and/or different limitations on angular positioning.

FIG. 8G shows an exemplary delivery system 880 having a flexible, optionally flaccid, extension 882. FIG. 8H shows an exemplary delivery system 890 having a gooseneck-type extension 892. These extensions may be integral or they may be selectively removable. In an exemplary embodiments of the invention, extensions 882 and 892 can bend 90°, 180°, 270° or even 360° or more degrees. Alternatively or additionally, the extensions can bend at two or more locations. In an alternative exemplary application, the delivery system is rigid and bent. A plurality of different degrees of bending and types of bending may be provided, for example, for access to various blood vessels inside the body, from outside the body, for example via keyholes.

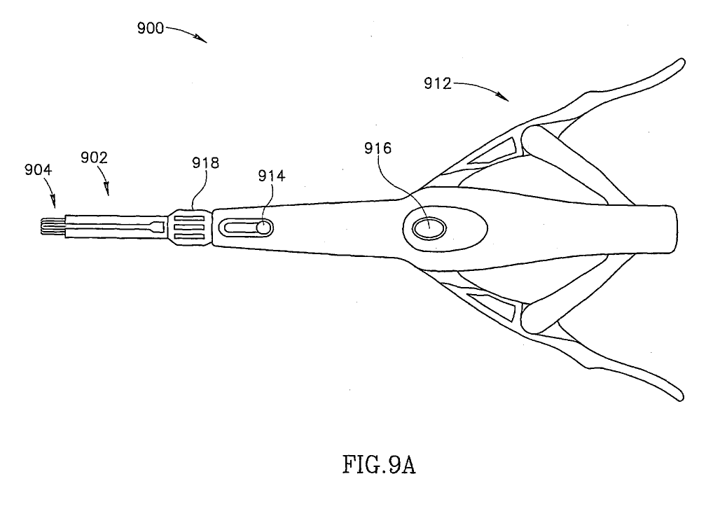

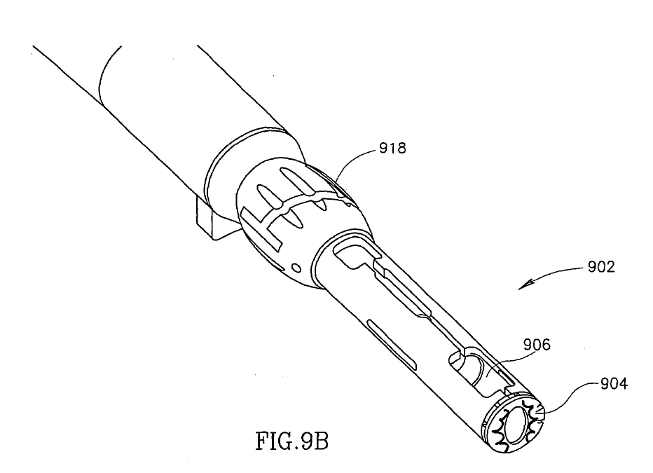

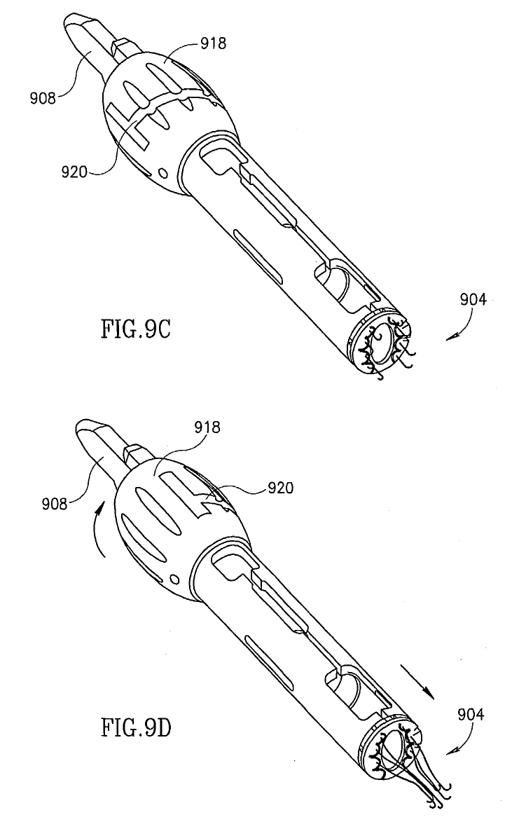

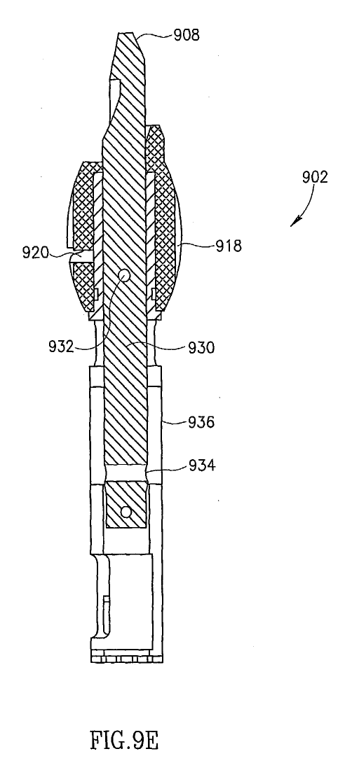

FIGS. 9A-9E illustrate an alternative embodiment of a capsule based delivery system 900, in accordance with an exemplary embodiment of the invention.

FIG. 9A shows system 900 including a handle 912, an optional safety switch 916 (e.g., to prevent motion of the handle and retraction of a connector), a capsule 902 and an optional locking lever 914 for selectively locking and releasing capsule 902 from system 900. An extension element (not shown) can be added between the body of system 900 and capsule 902.

FIG. 9B is an enlarged view of capsule 902 in a perspective view. A vessel opening 906 is provided for inserting a graft. The capsule as a whole is optionally splittable. A connector 904, for example one of the types described above, is schematically shown at a distal end of capsule 902. Unlike capsule 802, capsule 902 optionally includes a means for extending the forward spikes (not shown in this figure) of connector 904. In an exemplary embodiment of the invention, a rotating knob 918 is provided with at least one and optionally two locking positions, one with spikes extended and/or one with spikes retracted. Independently of this spike moving mechanism, the spikes as a whole may be retracted a considerable distance when handle 612 (FIG. 9A) is activated. Alternatively, knob 918 is used also to tear the spikes, as described above.

This selective extension of the spikes optionally serves to protect the spikes while guiding system 900 to an anastomosis location.

The operation of knob 918 is described with reference to FIGS. 9C-9E, in which FIGS. 9C and 9D show the outside of capsule 902 and FIG. 9E shows its inside mechanism. FIG. 9E shows a cut-through view of capsule 902, in accordance with an exemplary embodiment of the invention. Capsule 902 comprises, for example, an external shell 936 and an internal mandrel 930 including a proximal locking mechanism 908 for attaching to system 900. In an exemplary embodiment of the invention, mandrel 930 includes a peg 932 that interacts with a slot 920, for example a “Z” shaped slot, formed in knob 918. The sides of the “Z” define locking positions and the connecting line defines the motion of mandrel 930. In an exemplary embodiment of the invention, mandrel 930 includes a concentric depression 934 for receiving a spike section of a connector 904 (not shown). Mandrel 930 is optionally hollow for passage of a graft therethrough.

FIG. 9C shows capsule 902 in one position, with spikes of connector 904 being retracted. In FIG. 9D, knob 918 is rotated so that the spikes extend forward. In an exemplary embodiment of the invention, the motion is accompanied by retraction of shell 936.

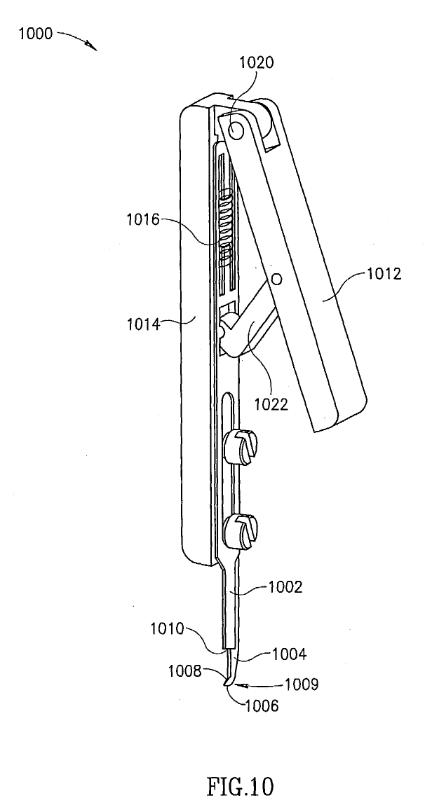

FIG. 10 illustrates an incision maker 1000 for forming an opening in a target vessel, from outside the blood vessel, in accordance with an exemplary embodiment of the invention. Two moving parts are provided, a base face 1010 coupled to a first handle 1014 and an ‘L” shaped spike 1004 coupled to a second handle 1012. In the exemplary embodiment shown, the two handles are connected using a hinge 1020 and an arm 1022. Other handle designs may be used, for example a syringe-like design and/or a gooseneck extension mechanism as described above. The two parts are optionally coupled using a spring 1016, or a spring in hinge 1020 (not shown). In an exemplary use, a tip 1006 of an arm 1009 of spike 1004 is inserted into a blood vessel, for example a coronary artery. Incision maker 1000 is then turned so that arm 1009 is inside the vessel and parallel to the vessel axis (assuming that is the desired cut direction, as an oblique cut or a trans-axial cut may be desired). Arm 1009 is then retracted towards face 1010 and the vessel wall is cut using a shearing cut. Optionally an inner face 1008 of arm 1009 is sharp and functions as a knife. In an exemplary embodiment of the invention, tip 1006 is flexible. Optionally, face 1010 is formed at the end of a flat plate 1002 and tip 1006 is bent inwards towards the plane of plate 1002.

Face 1010 and face 1008 are optionally substantially parallel to each other. Alternatively, the faces are not parallel to each other, for example, spreading out (as shown) or pointing in. One or both the faces may be perpendicular to the axis of motion incision maker 1000 or be oblique thereto.

The above description has focused on devices that are applied from outside a blood vessel. However, they can also be applied from inside of blood vessels.

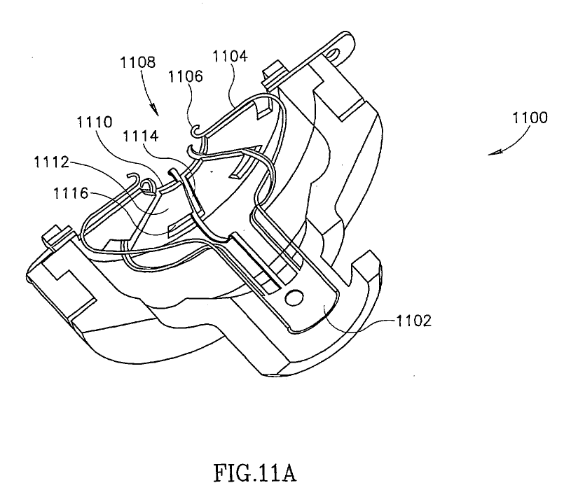

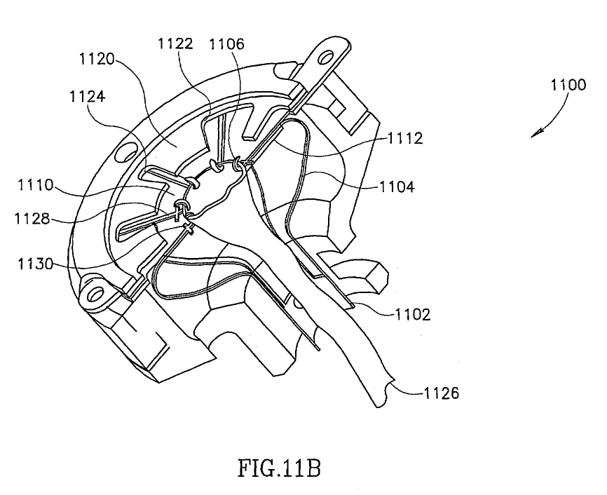

FIGS. 11A and 11B illustrate a connector 1102 and an associated graft mounting system 1100, in which the connector is distorted to assist in mounting a graft thereon, in accordance with an exemplary embodiment of the invention. Both these figures show connector 1102 already mounted in system 1100.

A connector 1102 comprises a plurality of extending spikes 1104, having recurved tips 1106. When the spikes point forward, mounting a graft 1126 on connector 1102 may be difficult, due to the direction of tips 1106. In an exemplary embodiment of the invention, mounting system 1100 bends spikes 1104 so that tips 1106 point radially out. Then graft 1126 can be mounted by providing the graft inside connector 1102 and pulling (e.g., with tweezers) the lip of graft 1126 over tips 1106 , to be impaled by tips 1106. In one example, the lip is everted manually over the tips one by one. Alternatively, a mechanical device may be used to grab the lip at a plurality of locations and extend it over the spike tips. Optionally, the spike tips are closer together than the graft diameter, to reduce the need to stretch the graft lip. (e.g., one by one).

System 1100 is shown in perspective in FIG. 11A from underneath and in FIG. 11B from its top. In general, system 1100 comprises three slotted disks 1120, 1110 and 1112. Spikes 1104 are held in slots 1128 formed in disk 1110. Optionally, the slots include a section of a tab-and-tear portion 1130 of each spike. In operation, all spikes 1104 are extended through an aperture 1108 formed in the disks. Disk 1112 is rotated and aligned with disk 1110, so that a plurality of slots 1114 formed in disk 1112 and especially a wide slot portion 1116 are aligned with slots 1130. In an exemplary embodiment of the invention, the slots are aligned to be partially overlapping, so that the overlapping portion is smaller than the width of a spike, but wider than a spike thickness. Each spike is twisted (e.g., using tweezers) and guided along the thus formed overlapping slot and allowed to untwist in wide slot portion 1116. Then, a plurality of slots 1122, 1124, etc. in disk 1120 are aligned in turn with the bent spikes. As shown in FIG. 11B, the distance between the slots and/or their width is non-uniform, allowing each spike to be dealt with in turn, when only its associated slot overlies a particular slot 1130. Each spike is pushed down into slot 1128. When this process is completed, graft 1126 may be mounted on the outward pointing sharp tips 1106. Once mounted, capsule 802 or 902 may be brought over the base of connector 1102. In an exemplary embodiment of the invention, connector 1102 is mounted first over a mandrel portion of the capsule and then the casing of the capsule is brought over the mandrel (e.g., as in FIG. 9). In an exemplary embodiment of the invention, the capsule design and/or delivery system is modified to account for a longer retraction of the connector, which longer retraction compensates for longer spikes that might be used in the embodiments of FIG. 11.

Although a particular example is shown, it should be appreciated that other disk patterns and alignment mechanism may be used as well for sequentially arranging the spikes in systems 1100. For example, different slot patterns or different motion of the disks may be provided.

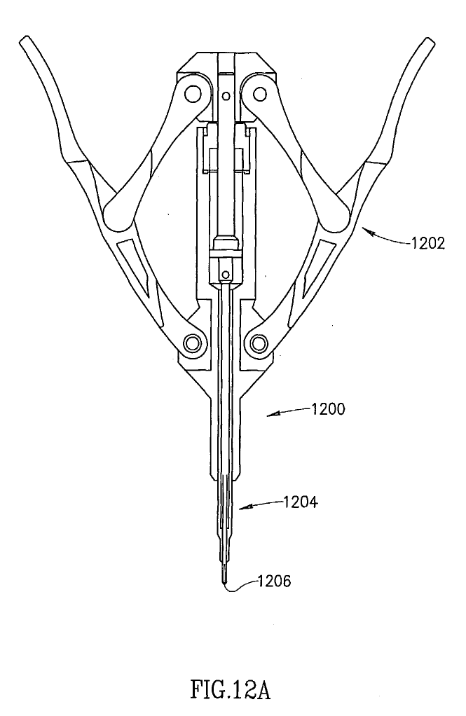

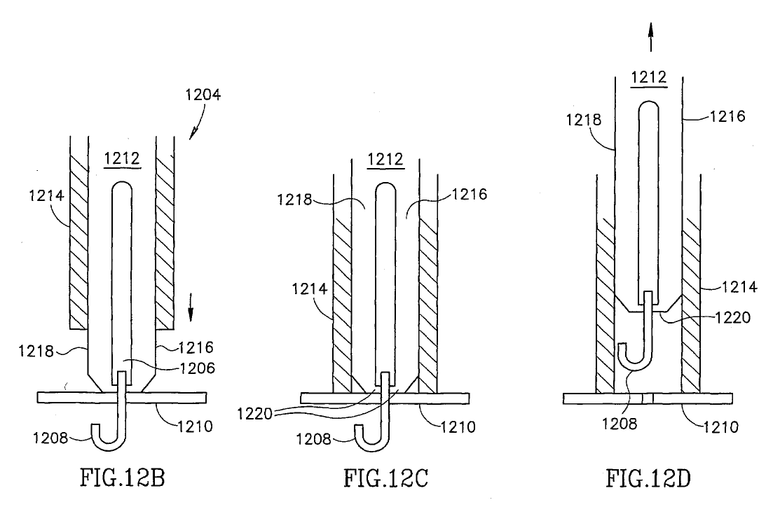

FIGS. 12A-12D illustrate a connector remover 1200, in accordance with an exemplary embodiment of the invention. Remover 1200 may be used to remove an anastomotic connector during implantation or after complete implantation. Generally, remover 1200 comprises a handle 1202 that retracts a tip 1206 relative to a body 1204.

FIGS. 12B-12D show remover 1200 is use. Tip 1026 comprises a fixed outer tube 1214 and a spike gripper, for example, a retractable split inner tube 1212 or a tweezers.

Tube 1212 is advanced until it meets a base ring portion 1210 of a connector (FIG. 12B), so that a spike 1208 extends into a hollow 1206 defined between two sides 1216 and 1218 of split tube 1212. In FIG. 12C, tube 1212 is retracted relative to tube 1214, while pressing the whole remover forward, thus the effect is that of advancing tube 1214 over tube 1212. In an exemplary embodiment of the invention, tube 1212 includes one or more protrusions 1220 at its distal end, that increase its diameter, tube 1212 has an increasing outer diameter at its distal end and/or outer tube 1214 has a narrowing inner diameter. Thus, when tube 1214 reaches base 1210, (or, possibly, before that time) spike 1208 is gripped by inner tube 1212. Continued retraction of inner tube 1212, for example as shown in FIG. 12D pulls spike 1208 through base ring portion 1210. In an embodiment where base ring 1210 includes a spring tab (e.g., 718, FIG. 7A) to prevent retraction of spike 1208 (under normal conditions), inner tube 1212 and/or outer tube 1214 are optionally designed to allow freedom of motion for the tab. Spike 1208 is optionally straightened by the retraction through base ring 1210.

Alternatively or additionally to friction engaging of the spike, in some embodiments of the invention, protrusions 1220 engage a tab portion or an aperture formed in the spike.

In another embodiment of the invention, a remover 1200 is adapted to remove a plurality or even all the spikes of a connector at one time. In this case, inner tube 1212 is optionally made of sufficient diameter to enclose the graft and the spikes are engaged between the inner tube and the outer tube. A separate further outer tube relative to which the two other tubes are retracted and which provides a contra against the base ring, may be provided.

Thus, in some embodiments of the invention, inner tube 1212 or a different type of spike gripper is not activated by a same overtube as used for applying a contra force against the base ring.

In an exemplary embodiment of the invention, the above devices are used in combination with anastomosis-related tools as described in PCT applications and publications WO 99/62415, WO 00/56226, WO 00/56228, WO 01/41623, WO 01/41624, PCT/IL01/00267, PCT/IL01/00069, PCT/IL01/00074, PCT/IL01/00266 and PCT/IL01/00600, the disclosures of which are incorporated herein by reference. However, they may also be used as stand alone devices or as part of surgical kits for other uses and/or anastomosis connectors.

It will be appreciated that the above described methods and devices of vascular manipulation may be varied in many ways, including, changing the order of steps, the exact materials used for the devices, which vessel is a “side” side and which vessel (or graft) is an “end” side of an end-to-side anastomosis and/or whether the end vessel is everted over the connector. Further, in the mechanical embodiments, the location of various elements may be switched, without exceeding the spirit of the disclosure, for example, switching the moving elements for non-moving elements where relative motion is required. In addition, a multiplicity of various features, both of methods and of devices have been described. It should be appreciated that different features may be combined in different ways. In particular, not all the features shown above in a particular embodiment are necessary in every similar exemplary embodiment of the invention. Further, combinations of the above features, from different described embodiments are also considered to be within the scope of some exemplary embodiments of the invention. In addition, some of the features of the invention described herein may be adapted for use with prior art devices, in accordance with other exemplary embodiments of the invention. The particular geometric forms used to illustrate the invention should not be considered as necessarily limiting the invention in its broadest aspect to only those forms, for example, where a circular lumen is shown, in other embodiments an oval lumen may be used.

Also within the scope of the invention are surgical kits which include sets of medical devices suitable for making a single or a small number of anastomosis connections and/or apertures. Measurements are provided to serve only as exemplary measurements for particular cases, the exact measurements applied will vary depending on the application. When used in the following claims, the terms “comprises”, “comprising”, “includes”, “including” or the like means “including but not limited to”.

It will be appreciated by a person skilled in the art that the present invention is not limited by what has thus far been described. Rather, the scope of the present invention is limited only by the following claims.

1. An anastomotic connector comprising:
a plurality of clip segments; and
a plurality of twistable resilient segments that interconnect the clip segments.

2. A connector according to claim 1, wherein said segments are bendable out of a plane defined by said clip segments.

3. A connector according to claim 1, wherein a resilience of said attachment segments is defined to control a diameter changing behavior of said connector.

4. A connector according to claim 1, wherein said clip segments do not penetrate target tissue when the clip closes.

5. A connector according to claim 1, wherein said clip segments do penetrate target tissue when the clip closes, but do not transfix said tissue.

6. An anastomotic connector comprising:
a plurality of clip segments each defining a clip contact area at which opposite sides of the clip engage tissue; and
a plurality of attachment segments that interconnect the clip segments, wherein said attachment segments lie in a first circumference and said contact areas lie in a second circumference and wherein said two circumferences are not the same.

7. A connector according to claim 6, wherein a resilience of said attachment segments is defined to control a diameter changing behavior of said connector.

8. A connector according to claim 6, wherein said circumferences are not on a same plane.

9. An anastomotic connector comprising:
a plurality of connection segments each defining a contact area between the segment and a target blood vessel; and
a plurality of attachment segments that interconnect said connection segments and limit relative motion of the connection segments, wherein some of said attachment segments limit relative motion of said connection segments more than the motion of other connection segments is limited.

10. A connector according to claim 9, wherein said connection segments each comprises an apertured segments through which a puller that engages the target vessel can be advanced and retracted.

11. A connector according to claim 9, comprising a base plate, wherein said connection segments each comprises an apertured area the base plate.

12. A connector according to claim 9, wherein said connection segments each comprises a clip element.

13. A connector according to claim 9, wherein the connector is designed to limit relative motion to a greater degree for areas of the target vessel that are expected to be under a higher degree of strain.

14. A connector according to claim 9, wherein said connection segments are arranged in the form of an ellipse and relative motion between segments is reduced at narrow ends of the ellipse.

15. An anastomosis connector comprising:
a base plate having a plurality of apertures defined therein; and
a set of pullers adapted to pass through at least some of said apertures during deployment of said connector,
wherein said base palate is made spatially sparse enough to allow suturing of an anastomotic connection, using a needle, through the base plate.

16. An anastomosis connector comprising:
a plurality of clip segments, each clip defining an engagement volume in which the clip engages tissue;
a plurality of attachment segments interconnecting the clip segments; and
a set of tissue pullers each including at least one tissue engager,
wherein, said clips define a plurality of apertures adapted for extending said tissue pullers through the apertures such that retracting the pullers carries engaged tissue into said engagement volume of said clips.

17. A connector according to claim 16, wherein said apertures are adapted for holding the connector using a connector holder.

18. A connector according to claim 16, wherein said tissue pullers comprises pairs of axially elongate pullers, each pair comprising two pullers co-axially disposed with respect to each other.

19. A connector according to claim 18, wherein one of said pair of connectors is adapted to engage tissue of a target blood vessel and wherein another of said pair of pullers is adapted to engage tissue of a vessel on which said connector is mounted prior to completing said anastomosis.

20. A connector according to claim 18, wherein at least one of said pair of pullers has a tip that is wider than said aperture, such that retracting the puller closes the clip when said clip has a resting point on either side of said aperture.

21. An anastomosis connector comprising:
a base plate defining a plurality of apertures therein; and
a set of pullers adapted to pass through at least some of said apertures during deployment of said connector, each of said pullers including a tissue engaging tip,
wherein said apertures are arranged in pairs, one aperture for receiving a puller and one aperture for receiving the tip of the puller when the puller is retracted.

22. A connector according to claim 21, comprising a bar around which said puller hooks, which bar defines a separation between said pair of apertures.

23. An anastomosis connector comprising:
a base plate defining a plurality of apertures therein; and
a set of pullers adapted to pass through at least some of said apertures during deployment of said connector,
wherein said each of said pullers comprises:
a tip; and
a tab section adapted to distort out of a plane of said puller when said puller is disconnected adjacent said tab.

24. A delivery system for an anastomosis connector, comprising:
a body; and
at least one slotted forward plate attached to said body, wherein said plate and said body define therebetween a receptacle for a base plate of an anastomosis connector and wherein said slots match apertures formed in said base plate for extension of tissue engagement spikes therethrough,
wherein said forward plate is adapted to bend out of the way of axial motion of said base plate, when said connector is deployed.

25. A system according to claim 24, wherein said forward plate is mounted on at least one hinge.

26. A system according to claim 24, wherein said forward plate is divided into at least two coplanar plates.

27. A system according to claim 24, wherein said forward plate is attached to said body via a distortable attachment.

28. A delivery system for an anastomosis connector, comprising:
a body including a handle for applying force;
a capsule adapted to interlock with said body and for carrying a connector, wherein said force is transferred by said interlocking to deploy said connector; and
an extension, adapted to be selectively connected between said body and said capsule, thereby extending a reach of said delivery system.

29. A system according to claim 28, wherein said extension is bendable.

30. A delivery system for an anastomosis connector, comprising:
a body including a handle for applying force;
a connector holder area defined at a distal end of said body and adapted for holding an anastomosis connector, wherein said force is transferred to said area for deploying said connector; and
a non-limp geometry changing elongate section bridging between said body and said area.

31. A system according to claim 30, wherein said elongate section is hinged.

32. A system according to claim 30, wherein said elongate section is distortable.

33. A system according to claim 30, comprising a flexible cable for transferring said force between said handle and said area.

34. A system according to claim 30, wherein said system is adapted for holding and deploying a two part connector comprising a plurality of tissue engaging elements that are retracted by said force during deployment, such that a base ring portion of the connector is engaged by the holder area and a tissue puller portion of the connector is retracted by said force.

35. A delivery system for an anastomosis connector, comprising:
a body including a handle for applying force;
a connector holder area defined at a distal end of said body and adapted for holding an anastomosis connector, wherein said force is transferred to said area for deploying said connector; and
a control for selectively advancing a plurality of tissue engaging elements from said connector holder area, said control being separate from said handle for applying force.

36. A system according to claim 35, wherein said control comprises a rotating knob.

37. A system according to claim 35, wherein said control is mounted on a separate capsule element that includes said connector holder area.

38. A system according to claim 35, wherein said tissue engaging elements form part of a connector.

39. A system according to claim 35, wherein said tissue engaging elements form part of said delivery system.

40. A connector removal device for removing a spike that completes an anastomotic connection in a spike and base type connector, comprising:
at least one spike engager adapted to engage at least one spike of a deployed connector;
an axially elongate contra element axially movable relative to said spike engager and adapted to rest against a base portion of said connector while said spike engager retracts said spike away from said base portion by said axial motion.

41. A device according to claim 40, wherein said spike engager engages said spike by friction.

42. A device according to claim 41, wherein said spike engager comprises a split tube.

43. A device according to claim 40, wherein said contra element comprises an overtube that fits over said spike engager and wherein said contra element has an inner diameter that clamps said spike engager shut when said engager is within said contra element.

44. A device according to claim 40, wherein said spike engager engages a plurality of spikes at a time.

45. A method of mounting a graft on a generally cylindrical connector having a plurality of axially extending spikes, each with a curved tip at a distal end of the connector, comprising:
bending each spike such that the spike points inwards towards the axis and the tip points outwards away from the axis;
bringing a graft to said axis between the inward pointing spikes; and
mounting the graft on the spike points.

46. A method according to claim 45, wherein bringing comprises conveying the graft between the spikes from a proximal end of the connector to the spike tips.

47. A method according to claim 46, wherein mounting comprises mounting the graft sequentially over the tips.

48. A method according to claim 45, wherein bending comprises mounting the connector in a jig.

49. A method according to claim 45, wherein bending comprises bending the spikes outwards and then bending inwards so that the bent part of the spikes define a diameter greater than that of unbent spikes.

50. A jig for holding a spiked connector in a configuration in which the spikes bend inwards, comprising;
a body defining an axial channel for receiving a connector; and
a plate attached to an end of said body and defining a plurality of trans-axial channels for receiving spikes of said connector, said plate defining a hole in its center for receiving a graft.

51. A jig according to claim 50, wherein said plate comprises a slotted disk.

52. A jig according to claim 50, comprises at least one rotatable disk underlying said plate for selectively defining slots along which said spikes can be conveyed when twisted.

53. A jig according to claim 50, comprises at least one rotatable slotted disk overlying said plate for selectively aligning slots of said disk with slots of said plate.

54. A jig according to claim 53, wherein said slots in said disk are arranged so that said selective alignment can be sequential for different ones of said slots of said plate.

55. A blood vessel cutter for forming an aperture in a blood vessel, comprising:
a first section having a cutting face; and
a second section having a cutting face matching said cutting face of said first section and axially movable relative thereto to provide shearing cutting action,
wherein, said second section comprises a pointed tip adapted for piercing a blood vessel; and
wherein said second section is flexible and said pointed tip is twisted towards a plane of said first section, such that when said first section is moved towards said second section, said first section pushes said second section out of its way.

56. A cutter according to claim 55, wherein said cutting face of said second section is sharp.

57. A cutter according to claim 55, wherein said cutting faces are not parallel to each other in a plane generally common to the two cutting faces.

58. A cutter according to claim 57, wherein said second cutting face is not perpendicular to said axial motion.

59. A cutter according to claim 57, wherein said pointed tip points away from said first plane.
